SEQUENCE LISTING (112 sequences)

SEQ ID NO: 1 - DNA, 28 bp, synthetic construct
  source 1..28: mol_type = other DNA
acataatactatctggatctacaccrga

SEQ ID NO: 2 - DNA, 28 bp, synthetic construct
  source 1..28: mol_type = other DNA
catttactgtattaatgctatcactagt

SEQ ID NO: 3 - DNA, 32 bp, synthetic construct
  source 1..32: mol_type = other DNA
cgacgtaccatacagacaccgtcacaacgtcg

SEQ ID NO: 4 - DNA, 27 bp, synthetic construct
  source 1..27: mol_type = other DNA
ctctctcattgatttttcgcgggatac

SEQ ID NO: 5 - DNA, 25 bp, synthetic construct
  source 1..25: mol_type = other DNA
acgatactcctcctcgttggtctac

SEQ ID NO: 6 - DNA, 32 bp, synthetic construct
  source 1..32: mol_type = other DNA
cgcgattaggccgtgtatcagcatccatcgcg

SEQ ID NO: 7 - DNA, 19 bp, synthetic construct
  source 1..19: mol_type = other DNA
gtcaacgctggaaggagtg

SEQ ID NO: 8 - DNA, 18 bp, synthetic construct
  source 1..18: mol_type = other DNA
ccagcagacagcctatcc

SEQ ID NO: 9 - DNA, 29 bp, synthetic construct
  source 1..29: mol_type = other DNA
ctcctgtactaaaaccacgwcaacaaact

SEQ ID NO: 10 - DNA, 16 bp, synthetic construct
  source 1..16: mol_type = other DNA
aatacaaggaggatct

SEQ ID NO: 11 - DNA, 18 bp, synthetic construct
  source 1..18: mol_type = other DNA
cttaactttttctttctc

SEQ ID NO: 12 - DNA, 18 bp, synthetic construct
  source 1..18: mol_type = other DNA
gagagaatctcttgatat

SEQ ID NO: 13 - DNA, 16 bp, synthetic construct
  source 1..16: mol_type = other DNA
attctagattgtaatc

SEQ ID NO: 14 - DNA, 18 bp, synthetic construct
  source 1..18: mol_type = other DNA
gagattagcagactccaa

SEQ ID NO: 15 - DNA, 21 bp, synthetic construct
  source 1..21: mol_type = other DNA
gattcaatttccagtttgtac

SEQ ID NO: 16 - DNA, 33 bp, synthetic construct
  source 1..33: mol_type = other DNA
gcagtcgttcaactgtatttcaagatctgagat

SEQ ID NO: 17 - DNA, 32 bp, synthetic construct
  source 1..32: mol_type = other DNA
ctagattgtaatctctgtagcatttccacggc

SEQ ID NO: 18 - DNA, 48 bp, synthetic construct
  source 1..48: mol_type = other DNA
ccgttaccgtttttacaatcgttaatcaatgctgatatggaaaagaga

SEQ ID NO: 19 - DNA, 47 bp, synthetic construct
  source 1..47: mol_type = other DNA
ataggctaaagactagaatcagggattctgattcatcctttgagaag

SEQ ID NO: 20 - DNA, 18 bp, synthetic construct
  source 1..18: mol_type = other DNA
tacagttgaacgactgcg

SEQ ID NO: 21 - DNA, 22 bp, synthetic construct
  source 1..22: mol_type = other DNA
agttcagttttatatgccgaat

SEQ ID NO: 22 - DNA, 25 bp, synthetic construct
  source 1..25: mol_type = other DNA
gatgtctatcaagatccatgattct

SEQ ID NO: 23 - DNA, 20 bp, synthetic construct
  source 1..20: mol_type = other DNA
tcttgaacgatcgctagaga

SEQ ID NO: 24 - DNA, 50 bp, synthetic construct
  source 1..50: mol_type = other DNA
tggagtctgctaatctctgtaagattagagaactagagaataagttgacc

SEQ ID NO: 25 - DNA, 39 bp, synthetic construct
  source 1..39: mol_type = other DNA
tgagtgaatgccgtggaaatgcgcagtcgttcaactgta

SEQ ID NO: 26 - DNA, 20 bp, synthetic construct
  source 1..20: mol_type = other DNA
cacaagaagttgatgcactg

SEQ ID NO: 27 - DNA, 23 bp, synthetic construct
  source 1..23: mol_type = other DNA
cagcattgatttcattattacgt

SEQ ID NO: 28 - DNA, 16 bp, synthetic construct
  source 1..16: mol_type = other DNA
cgctctcgatgcagtc

SEQ ID NO: 29 - DNA, 25 bp, synthetic construct
  source 1..25: mol_type = other DNA
cagagattacaatctagaatctcag

SEQ ID NO: 30 - DNA, 23 bp, synthetic construct
  source 1..23: mol_type = other DNA
ccagagatatcatagccgctctt

SEQ ID NO: 31 - DNA, 24 bp, synthetic construct
  source 1..24: mol_type = other DNA
gaaactctcaaacaacgrctaact

SEQ ID NO: 32 - DNA, 19 bp, synthetic construct
  source 1..19: mol_type = other DNA
taaatagaacgtcatcatt

SEQ ID NO: 33 - DNA, 19 bp, synthetic construct
  source 1..19: mol_type = other DNA
atgacacaattaccaatac

SEQ ID NO: 34 - DNA, 17 bp, synthetic construct
  source 1..17: mol_type = other DNA
ctagactttgttctctg

SEQ ID NO: 35 - DNA, 17 bp, synthetic construct
  source 1..17: mol_type = other DNA
ctgataatgtagaagac

SEQ ID NO: 36 - DNA, 17 bp, synthetic construct
  source 1..17: mol_type = other DNA
ttgtatttacgtgggtg

SEQ ID NO: 37 - DNA, 22 bp, synthetic construct
  source 1..22: mol_type = other DNA
gatgatgcaactctatcatgta

SEQ ID NO: 38 - DNA, 26 bp, synthetic construct
  source 1..26: mol_type = other DNA
gtataattatcaaaatacaagacgtc

SEQ ID NO: 39 - DNA, 18 bp, synthetic construct
  source 1..18: mol_type = other DNA
agtgcttggtataaggag

SEQ ID NO: 40 - DNA, 28 bp, synthetic construct
  source 1..28: mol_type = other DNA
attggtcattatttttgtcacaggaaca

SEQ ID NO: 41 - DNA, 23 bp, synthetic construct
  source 1..23: mol_type = other DNA
aatggcgttgacaattatgggtg

SEQ ID NO: 42 - DNA, 14 bp, synthetic construct
  source 1..14: mol_type = other DNA
agagattagaaata

SEQ ID NO: 43 - DNA, 22 bp, synthetic construct
  source 1..22: mol_type = other DNA
acgtgttaaacaatgggtgatg

SEQ ID NO: 44 - DNA, 23 bp, synthetic construct
  source 1..23: mol_type = other DNA
aacatttccatgaatcgtagtcc

SEQ ID NO: 45 - DNA, 28 bp, synthetic construct
  source 1..28: mol_type = other DNA
tgaatgaatgcgatactgtatgtgtggg

SEQ ID NO: 46 - DNA, 43 bp, synthetic construct
  source 1..43: mol_type = other DNA
tgggagcattgtaacttatagttgccctcctgaacacatgaca

SEQ ID NO: 47 - DNA, 44 bp, synthetic construct
  source 1..44: mol_type = other DNA
atcctcgtatccgttatgtcttcccacctatttgcgaatctgtt

SEQ ID NO: 48 - DNA, 18 bp, synthetic construct
  source 1..18: mol_type = other DNA
tgggtggattggaccatt

SEQ ID NO: 49 - DNA, 19 bp, synthetic construct
  source 1..19: mol_type = other DNA
atggtatggaatcctgagg

SEQ ID NO: 50 - DNA, 25 bp, synthetic construct
  source 1..25: mol_type = other DNA
gatattcgttgattggtaactctgg

SEQ ID NO: 51 - DNA, 25 bp, synthetic construct
  source 1..25: mol_type = other DNA
gttggatatagatggaggtgattgg

SEQ ID NO: 52 - DNA, 24 bp, synthetic construct
  source 1..24: mol_type = other DNA
tgtctacctggatacagaaagcaa

SEQ ID NO: 53 - DNA, 24 bp, synthetic construct
  source 1..24: mol_type = other DNA
ggcatctccgtttaatacattgat

SEQ ID NO: 54 - DNA, 30 bp, synthetic construct
  source 1..30: mol_type = other DNA
cccatatatgctaaatgtaccggtaccgga

SEQ ID NO: 55 - DNA, 23 bp, synthetic construct
  source 1..23: mol_type = other DNA
tcaactgaaaaggccatctatga

SEQ ID NO: 56 - DNA, 28 bp, synthetic construct
  source 1..28: mol_type = other DNA
gagtatagagcactatttctaaatccca

SEQ ID NO: 57 - DNA, 32 bp, synthetic construct
  source 1..32: mol_type = other DNA
ccatgcaatatacgtacaagatagtagccaac

SEQ ID NO: 58 - DNA, 22 bp, synthetic construct
  source 1..22: mol_type = other DNA
ctcattgatttttcgcgggata

SEQ ID NO: 59 - DNA, 22 bp, synthetic construct
  source 1..22: mol_type = other DNA
gacgatactcctcctcgttggt

SEQ ID NO: 60 - DNA, 20 bp, synthetic construct
  source 1..20: mol_type = other DNA
catcagaatctgtaggccgt

SEQ ID NO: 61 - DNA, 25 bp, synthetic construct
  source 1..25: mol_type = other DNA
catctattatagcatcagcatcaga

SEQ ID NO: 62 - DNA, 23 bp, synthetic construct
  source 1..23: mol_type = other DNA
gatactcctcctcgttggtctac

SEQ ID NO: 63 - DNA, 25 bp, synthetic construct
  source 1..25: mol_type = other DNA
tgtaggccgtgtatcagcatccatt

SEQ ID NO: 64 - DNA, 20 bp, synthetic construct
  source 1..20: mol_type = other DNA
cacaccgtctcttccacaga

SEQ ID NO: 65 - DNA, 23 bp, synthetic construct
  source 1..23: mol_type = other DNA
gatacaggttaatttccacatcg

SEQ ID NO: 66 - DNA, 26 bp, synthetic construct
  source 1..26: mol_type = other DNA
aacccgtcgtaaccagcaatacattt

SEQ ID NO: 67 - DNA, 26 bp, synthetic construct
  source 1..26: mol_type = other DNA
ggaaaatgtaaagacaacgaatacag

SEQ ID NO: 68 - DNA, 24 bp, synthetic construct
  source 1..24: mol_type = other DNA
gctatcacataatctggaagcgta

SEQ ID NO: 69 - DNA, 30 bp, synthetic construct
  source 1..30: mol_type = other DNA
aagccgtaatctatgttgtctatcgtgtcc

SEQ ID NO: 70 - DNA, 32 bp, synthetic construct
  source 1..32: mol_type = other DNA
aataaacggaagagatatagcaccacatgcac

SEQ ID NO: 71 - DNA, 29 bp, synthetic construct
  source 1..29: mol_type = other DNA
gtgagatgtaaaggtatccgaaccacacg

SEQ ID NO: 72 - DNA, 29 bp, synthetic construct
  source 1..29: mol_type = other DNA
acagaagccgtaatctatgttgtctatcg

SEQ ID NO: 73 - DNA, 26 bp, synthetic construct
  source 1..26: mol_type = other DNA
aacaaccgtcctacaattaaacaaca

SEQ ID NO: 74 - DNA, 25 bp, synthetic construct
  source 1..25: mol_type = other DNA
cgctatcgaaccatttttgtagtct

SEQ ID NO: 75 - DNA, 21 bp, synthetic construct
  source 1..21: mol_type = other DNA
tataacggcgaagaatatact

SEQ ID NO: 76 - DNA, 100 bp, synthetic construct
  source 1..100: mol_type = other DNA
ccaatggaaaatgtaaagacaacgaatacagaagccgtaatctatgttgtctatcgtgtcctccgggaacttacgcttccagattatgtgatagcaagac

SEQ ID NO: 77 - DNA, 100 bp, synthetic construct
  source 1..100: mol_type = other DNA
ccaatggaaagtgtaaagacaacgaatacagaagccgtaatctatgttgtctatcgtgtcctccgggaacttacgcttccagattatgtgatagcaagac

SEQ ID NO: 78 - DNA, 100 bp, synthetic construct
  source 1..100: mol_type = other DNA
ccaatggaaagtgtaaagacaccgaatacaaacgccataatctgtgttgtttatcgtgtcctccgggaacatacgcttccagattatgtgatagcaagac

SEQ ID NO: 79 - DNA, 100 bp, synthetic construct
  source 1..100: mol_type = other DNA
ccaatggaaagtgtaaagacaacgaatacaaacgccataatttgtgttgtttatcgtgtcctccgggaacatacgcttccagattatgcgatagcaagac

SEQ ID NO: 80 - DNA, 100 bp, synthetic construct
  source 1..100: mol_type = other DNA
ccaacggaaagtgtaaagacaacgaatacaaacgccatcatctatgttgtttatcgtgtcctccgggaacatacgcttccagattatgcgatagcaagac

SEQ ID NO: 81 - DNA, 100 bp, synthetic construct
  source 1..100: mol_type = other DNA
ccaatggaaagtgtaaagacaacgaatacaaacgccataatctgtgttgtttatcgtgtcctccgggaacatacgcttccagattatgcgatagcaagac

SEQ ID NO: 82 - DNA, 85 bp, synthetic construct
  source 1..85: mol_type = other DNA
ccgatggaaagtgtaaagacaacgaatacaaacgccataatttgtgtccgggaacatacgcttccagattatgcgatagcaagac

SEQ ID NO: 83 - DNA, 85 bp, synthetic construct
  source 1..85: mol_type = other DNA
ccgatggaaagtgtaaagacaacgaatacaaacgccataatttgtgtccgggaacatacgcttccagattatgcgatagcaaaac

SEQ ID NO: 84 - DNA, 100 bp, synthetic construct
  source 1..100: mol_type = other DNA
ccaatggaaagtgtaaagccaacgaatacagaagccataatctatgttgtctatcgtgtcctccgggaacatacgcttccagattatgtgatagcgagac

SEQ ID NO: 85 - DNA, 100 bp, synthetic construct
  source 1..100: mol_type = other DNA
ttaatgggaaatgtaacggtacagactataacagtaataatctatgttgtaaacaatgcaatcctggaatgtatatgactcattcctgtaataccacttc

SEQ ID NO: 86 - DNA, 100 bp, synthetic construct
  source 1..100: mol_type = other DNA
ccaatggaaagtgtaaagacaacgaatacaaacgccataatctgtgttgtttatcgtgtcctccgggaacatacgcttccagattatgcgatatcaagac

SEQ ID NO: 87 - DNA, 100 bp, synthetic construct
  source 1..100: mol_type = other DNA
ccaatggaaagtgtaaagacaacgaatacagacgccataatctatgttgtctatcgtgtcctccgggaacctacgcttccagattatgtgatagcaagac

SEQ ID NO: 88 - DNA, 100 bp, synthetic construct
  source 1..100: mol_type = other DNA
tagatggaaagtgcaaaggaggcgactacaagaatggtaatctttgttgtagctcttgctcggcgggaacatacgcttctaagctatgcgattccagtaa

SEQ ID NO: 89 - DNA, 100 bp, synthetic construct
  source 1..100: mol_type = other DNA
cgaatggaaagtgtaaaaacggcgaatataagcgaggtaatcaatgctgtaacagttgtccggcaggaacatacgcttctaaactatgcgatggtacaac

SEQ ID NO: 90 - DNA, 100 bp, synthetic construct
  source 1..100: mol_type = other DNA
ctaatggaaagtgtaaagaacacgaatacaagcgaggtaatcaatgttgtatcagatgtccagcgggaacatacgcttctaaacaatgcgatggttccac

SEQ ID NO: 91 - DNA, 120 bp, synthetic construct
  source 1..120: mol_type = other DNA
tgatcctctctcattgatttttcgcgggatacatcatctattatagcatcagcatcagaatctgtaggccgtgtatcagcatccattgtcgtagaccaacgaggaggagtatcgttggag

SEQ ID NO: 92 - DNA, 120 bp, synthetic construct
  source 1..120: mol_type = other DNA
ttatcctctctcattgatttttcgcgggatacatcatctattatagcatcagcatcagaatctgtaggccgtgtatcagcatccattgtcgtagaccaacgaggaggagtatcgtcggaa

SEQ ID NO: 93 - DNA, 120 bp, synthetic construct
  source 1..120: mol_type = other DNA
tgatcatctctcattgatttttctcgggatacatcatctattatatcatcagcatcagaatctgtaggctgtgtatcagcatccattgtcgtaaaccaacgaggaggagtatcgtcggag

SEQ ID NO: 94 - DNA, 120 bp, synthetic construct
  source 1..120: mol_type = other DNA
tgatcctctctcattgatttttcgcgggatacatcatctattatgacgtcagccatagcatcagtatccggcttatccgcctccgttgtcataaaccaacgaggaggaatatcgtcggag

SEQ ID NO: 95 - DNA, 120 bp, synthetic construct
  source 1..120: mol_type = other DNA
tgatcctctctcattgatttttcgcgggatacatcatctattatggcatcagccataacatcagcatccggcttatctgcctccattgtcataaaccaacgaggaggaatatcgtcggag

SEQ ID NO: 96 - DNA, 120 bp, synthetic construct
  source 1..120: mol_type = other DNA
tgatcctctctcattgatttttcgcgggatacatcatctattatgacgtcagccatagcatcagcatccggcttatccgcctccgttgtcataaaccaacgaggaggaatatcgtcggag

SEQ ID NO: 97 - DNA, 120 bp, synthetic construct
  source 1..120: mol_type = other DNA
tgatcctctctcattgatttttcgcgggatacatcatctattatgacgtcagccatagcatcagcattcggcttatccgcctccgttgtcataaaccaacgaggaggaatatcgtcggag

SEQ ID NO: 98 - DNA, 120 bp, synthetic construct
  source 1..120: mol_type = other DNA
tgatcctctctcattgatttttcgcgggatacatcatctattatggcgtcagccataacatcagcatccggcttatccgcctccgttgtcataaaccaacgaggaggaatatcgtcggag

SEQ ID NO: 99 - DNA, 107 bp, synthetic construct
  source 1..107: mol_type = other DNA
ctcattgatttttcgcgggatacatcatctattatagcatcagcatcagaatctgtaggccgtgtatcagcatccattgtcgtagaccaacgaggaggagtatcgtc

SEQ ID NO: 100 - DNA, 107 bp, synthetic construct
  source 1..107: mol_type = other DNA
ctcattgatttttcgcgggatacatcatctattatagcatcagcatcagaatctgtaggctgtgtatcagcatccattgtcgtaaaccaacgaggaggagtatcgtc

SEQ ID NO: 101 - DNA, 107 bp, synthetic construct
  source 1..107: mol_type = other DNA
ctcattgatttttcgcgggatacatcatctattatagcatcagcatcagaatctgtaggctgtgaatcagcatccattgtcgtaaaccaacgaggaggaatatcgtc

SEQ ID NO: 102 - DNA, 107 bp, synthetic construct
  source 1..107: mol_type = other DNA
ctcattgatttttcgcgggatgcatcatctattatagcatcagcatcagaatctgtaggctgtgaatcagcatccattgtcgtaaaccaacgaggaggaatatcgtc

SEQ ID NO: 103 - DNA, 174 bp, synthetic construct
  source 1..174: mol_type = other DNA
acataatactatctggatctacaccagaaactatttctgagaaaccagaggatatagataattctaattgctcgtctgtattcgaaatcacgactccggaaccaattactgataatgtagacgaccatacagacaccgtcacatacactagtgatagcattaatacagtaaatg

SEQ ID NO: 104 - DNA, 174 bp, synthetic construct
  source 1..174: mol_type = other DNA
acataatactatctggatctacaccggaaactatttctgagaaaccagaggatatagataattctaattgctcgtctgtattcgaaatcgcgactccggaaccaattactgataatgtagaagaccatacagacaccgtcacatacactagtgatagcattaatacagtaaatg

SEQ ID NO: 105 - DNA, 180 bp, synthetic construct
  source 1..180: mol_type = other DNA
acataatactatctggatctacacattcaccagaaactagttctgagaaaccagaggatatagataattctaattgctcgtcggtattcgaaatcgcgactccggaaccaattactgataatgtagaagatcatacagacaccgtcacatacactagtgatagcattaatacagtaagtg

SEQ ID NO: 106 - DNA, 180 bp, synthetic construct
  source 1..180: mol_type = other DNA
acataatactatctggatctacacattcaccagaaactagttctgagaaaccagaggatatagataattttaattgctcgtcggtattcgaaatcgcgactccggaaccaattactgataatgtagaagatcatacagacaccgtcacatacactagtgatagcattaatacagtaagtg

SEQ ID NO: 107 - DNA, 180 bp, synthetic construct
  source 1..180: mol_type = other DNA
acataatactatctggatctacacattcaccagaaactagttctgagaaacctgattatatagataattctaattgctcgtcggtattcgaaatcgcgactccggaaccaattactgataatgtagaagatcatacagacaccgtcacatacactagtgatagcattaatacagtaagtg

SEQ ID NO: 108 - DNA, 180 bp, synthetic construct
  source 1..180: mol_type = other DNA
acataatactatctggatcttcacattcaccggaaactagttctgagaaacctgattatataaataattttaattgctcgttggtatttgaaatcgcgactccgggaccaattactgataatgtagaaaatcatacagacactgtcacatacactagtgatatcattaatacagtaagta

SEQ ID NO: 109 - DNA, 177 bp, synthetic construct
  source 1..177: mol_type = other DNA
acataatactatctggatctacacattcaccggaaactagttctgagaaacctgattatatagataattctaattgctcgtcggtattcgaaatcgcgactccggaaccaattactgataatgaagaagatcacactgacgtcacatatactagtgaaaatattaatacagtaagta

SEQ ID NO: 110 - DNA, 180 bp, synthetic construct
  source 1..180: mol_type = other DNA
acataatactatctggatctacacattcactggaaactatttctgagaaaccagaggatatagataattctaattgctcgtctgtattcgaaatcgcgactccggaaccaattactgataatgtagaagatcatacagacactgtcacatacactagtgatatcattaatacagtaagta

SEQ ID NO: 111 - DNA, 107 bp, synthetic construct
  source 1..107: mol_type = other DNA
ctcattgatttttcgcgggatacatcatctattatagcatcagcatcagaatctgtaggccgtgtatcagcatccattgtcgtagaccaacgaggaggagtatcgtt

SEQ ID NO: 112 - DNA, 14 bp, synthetic construct
  source 1..14: mol_type = other DNA
cctccgggaactta

(19) United States
(12) Patent Application Publication
(10) Pub. No.: US 2026/0125771 A1
(43) Pub. Date: May 7, 2026

(54) MPX-HA Assay For Real-Time Detection Of Monkeypox

(71) Applicant: Hackensack Meridian Health, Inc., Edison, NJ (US)

(72) Inventors: Yanan Zhao, Livingston, NJ (US); David S. Perlin, New York, NY (US)

(73) Assignee: Hackensack Meridian Health, Inc, Edison, NJ (US)

(21) Appl. No.: 19/117,900

(22) PCT Filed: Oct. 3, 2023
(86) PCT No.: PCT/US2023/075844

§ 371 Date: Apr. 2, 2025

Related U.S. Application Data
(60) Provisional application No. 63/413,089, filed on Oct. 4, 2022.

(51) Int. Cl. C12Q 1/70

(52) U.S. Cl. CPC C12Q 1/701; C12Q 2600/156

(57) ABSTRACT

Disclosed are an assay, method, and kit for detecting a monkeypox virus (MPXV) and the application thereof. The assay may be used alone or in combination with another assay or assays. The method of using and kit includes a composition used for detecting MPXV, wherein the composition is composed of a primer pair and a probe. The primer pair is composed of two single-stranded nucleic acid molecules shown in the first sequence and the second sequence in a sequence list, and the sequence of the probe is the third sequence in the sequence list. The kit is used for detecting MPXV, and has advantages of providing real-time screening of MPXV reducing cross-reaction to other pox diseases, and increasing specificity to MPXV for disease prevention and improvement of public health.

### CROSS REFERENCE TO RELATED APPLICATIONS

This application claims priority from U.S. provisional application No. 63/413,089, filed Oct. 4, 2022, the disclosures of which are hereby incorporated herein by reference.

### TECHNICAL FIELD

The present disclosure relates to the biological technical field relating to an assay, method, and test kit for detecting monkeypox virus (MPXV) and application thereof.

The present disclosure also relates to an assay, or combination of assays, method, and test kit that are specific for MPXV without cross reaction to other pox viruses that allow real-time detection of monkeypox virus.

### BACKGROUND

The emergence of disease-causing viruses is a constant threat to humanity as each year novel viruses are being found (Woolhouse et al., 2012). Unprecedented global changes—population growth, increased trade and travel, and climate change—in the recent decades have made the threat of re-emergence of viruses even more likely (Baker et al., 2022).

The recent emergence of monkeypox disease by monkeypox virus (MPXV) has made global headlines. In May 2022, over 92 monkeypox cases were reported from 12 countries (WHO, 2022a). Although there was a major outbreak of monkeypox in the United States in 2003 via prairie dogs infected from an imported Gambian pouched rat (Karem et al., 2005; Reed et al., 2004), the recent unrelated outbreaks in multiple countries have caused serious concerns post-COVID and pressed for the need of disease surveillance and viral detection.

Indeed, the recent widespread emergence of monkeypox, a rare and endemic zoonotic disease by monkeypox virus (MPXV), has made global headlines. While transmissibility (R0≈0.58) and fatality rate (0-3%) are low, its prolonged morbidity of two to four weeks, and lengthy incubation period of up to 21 days has caused monkeypox to become a considerable public health concern. Thus, effective containment and disease management require quick and efficient detection of MPXV.

Monkeypox virus is a double-stranded DNA virus, Poxviridae, that may be categorized together with variola virus. The symptoms to a person infected with MPXV is similar to smallpox, such as body temperature increase or a fever, headache, lymphadenectasis, cough and extreme whole-body pain. Compared to smallpox, monkeypox has a much lower infectivity or human to human transmission with R0<1 (Learned et al., 2005; Sklenovská and Van Ranst, 2018), perhaps ≈0.576 (McMullen, 2015), and an attack rate of ≈50% (Luciani et al., 2021). While antiviral drug ST-246, a potent Orthopoxvirus egress inhibitor, can protect non-human primates from MPXV (Huggins et al., 2009), smallpox vaccine (for example, Dryvax) seems 85% effective in humans against monkeypox (Edghill-Smith et al., 2005). The MPXV has two distinct clades (Likos et al., 2005). The Congo basin clade causes illness similar to smallpox and has a case fatality rate of up to 10% in unvaccinated populations. The West African clade causes less severe and less interhuman transmissible disease (Chen et al., 2005; Li et al., 2010). The disease severity of monkeypox, compared to smallpox, is considerably less with milder rash.

Prior to the 2022 outbreak, monkeypox was mostly endemic in West and Central African countries. However, monkeypox cases are rising at an alarming rate worldwide since May 2022, with over 25,600 confirmed cases in the US and 68,000 globally as of Sep. 7, 2022. See for example: (https://www.cdc.gov/poxvirus/monkeypox/response/2022/world-map.html).

Diagnosis for human orthopoxvirus infections is largely limited to polymerase chain reaction (PCR) based molecular testing. As part of public health preparedness for infectious disease threats, the Center for Disease Control (CDC) has made diagnostic testing for orthopoxviruses available at Laboratory Response Network (LRN).

More recently, the CDC developed a non-variola Orthopoxvirus (NVO) real-time PCR diagnostic test (with FDA 510(k) clearance) to facilitate monkeypox detection. However, this NVO test does not differentiate monkeypox virus from other NVOs. To date, there is currently no commercially available assay to specifically detect monkeypox virus.

Therefore, a detection technique or assay to specifically detect monkeypox virus has great importance. There is a need for an improved assay, kit, and method for real-time detection of the monkeypox virus before any symptomatology arises. There is also a need to have such a detection without cross reaction to other pox virus such as cowpox and the like.

### SUMMARY

Compared to the above prior attempts, the present disclosure fulfills the above criteria and provides additional benefits that state of the art systems cannot provide.

The following monkeypox specific real-time PCR assays can be used as a sensitive and accurate tool for monkeypox diagnosis. It is based on detection the detection of MPXV using an assay of MPX-HA with a specialized molecular beacon and, depending on the implementation, this MPX-HA assay may also be combined with another assay of MPX-F3L for early detection of monkeypox before any lesions are formed.

One aspect of the present invention is to provide a composition for detecting monkeypox virus. The present invention includes a newly created, molecular beacon (MB) based HA real-time PCR assay, taking advantage of the single nucleotide mismatch (SNP) recognition feature of MBs. This assay is composed of a forward primer, a reverse primer generating a 174-bp amplicon, and a HA MB probe for MPX-specific detection.

The present novel MPX-HA assay is made up of a MB probe and two primers. For the primers, the proteins have the following sequence of amino acids ACATAATACTATCTGGATCTACACCRGA [SEQ. 1] for the forward primer, and CATTTACTGTATTAATGCTATCACTAGT [SEQ. 2] for the reverse primer, respectively, as shown in sequence Table I. The sequence for the described specialized probe includes the following probe sequence for MPX-HA, namely FAM-CGACGTACCATACAGACACCGTCACAACGTCG-Dabcyl [SEQ. 3] as shown for example in Table I.

To avoid cross reaction with other pox viruses such as cowpox, the design of the MPX-specific HA MB probe ensures that at a selected annealing temperature, the probe remains closed and dark in the presence of the single mismatch target, while it tightly binds to its perfect target resulting in bright fluorescence as a detection signal.

In another aspect of the invention, the assay for MPX-HA may be combined with other MPXV assays such as the disclosed MPX-F3L assay. The presently described specialized MPX-F3L assay or second assay is made up of a novel second MB probe and a second forward primer and a second reverse primer. For the second primers, the proteins have the following sequence of amino acids CTCTCTCATTGATTTTTCGCGGGATAC [SEQ. 4] for the forward primer, and ACGATACTCCTCCTCGTTGGTCTAC [SEQ. 5] for the reverse primer, respectively, as shown in sequence Table II.

The sequence for the described specialized second probe for the MPX-F3L assay includes the following FAM-CGCGATTAGGCCGTGTATCAGCATCCATCGCG-Dabcyl [SEQ. 6] as also shown in Table II.

In another aspect of the present invention is to provide a test kit for detecting monkeypox virus. The test kit for detecting monkeypox virus provided by the present invention, may include the following (a) or (b):
(a) containing described MPX-HA assay composition and/or assay combination of MPX-HA and MPX-F3L assays, and following material: Premix Ex Taq® a 2× master mix for real-time PCR (qPCR) using probe-based qPCR assays. This 2× master mix includes TaKaRa Ex Taq® HS—a hot-start PCR enzyme that uses an anti-Taq antibody—in a qPCR-optimized buffer. TaKaRa Ex Taq® HS inhibits non-specific amplification while enabling high-efficiency amplification and detection sensitivity during real-time PCR analyses. Additionally, Tli RNase H, a heat-resistant RNase enzyme, is included in the real-time PCR premix in order to minimize PCR inhibition due to the presence of residual mRNA in the input cDNA. This master mix is ideal for high-speed PCR, allows accurate target quantification and detection over a broad dynamic range, and enables highly reproducible and reliable real-time PCR analyses. A DNA template comprising synthetic oligo targets and/or monkeypox genomic DNA templates are included.
(b) containing described primer pair and probe for MPX-HA and/or combination of MPX-HA and MPX-F3L assays, and following material: 10 μl of Premix Ex Taq (Probe qPCR) (Takara manf. Cat #RR390L), 0.8 μl of 1 μM forward primer, 2 μl of 10 μM reverse primer, 0.4 μl of 5 μM MPX-HA MB probe, and 5 μl of DNA template-synthetic oligo targets and/or monkeypox genomic DNA templates are included.

In yet another aspect of the present invention, a method of using the inventive assay or combination of assays in the detection of MPXV. The steps in one embodiment, include, but are not limited to the following. The method of using the MPX-HA assay includes setting up in a 20 μl reaction material comprising of 10 μl of Premix Ex Taq (Probe qPCR) (TaKaRa Cat #RR390L), 0.8 μl of 1 μM forward primer, 2 μl of 10 μM reverse primer, 0.4 μl of 5 μM MPX-HA MB probe, and 5 μl of DNA template. Reactions are running on the Bio Molecular System mic qPCR cycler, with thermal profile of 95° C. for 2 min, 50 cycles of 95° C. for 5 sec and 56° C. for 20 sec, followed by 95° C. for 20 sec, then melted from 53° C. to 63° C. with a ramp rate of 0.1° C./s. This MPX-HA assay takes 1 hour and 7 min to complete.

The steps for the MPX-F3L assay includes setting up in a 20 μl reaction material comprising of 10 μl of Premix Ex Taq (Probe qPCR) (TaKaRa Cat #RR390L), 0.4 μl of 10 μM forward primer, 0.4 μl of 10 μM reverse primer, 0.4 μl of 5 μM MPX-F3L MB probe, and 5 μl of DNA template. Reactions are running on the Bio Molecular System mic qPCR cycler, with thermal profile of 95° C. for 2 min followed by 40 cycles of 95° C. for 5 sec and 60° C. for 20 sec. The real-time PCR test takes at least 48 min to complete.

Depending on the embodiment, either the MPX-HA assay alone or in combination with the MPX-F3L assay may be utilized to detect the MPXV virus.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

To assist those of skill in the art in making and using the disclosed composition and method, reference is made to the accompanying figures, wherein:

FIG. 1 Prior Art as shown illustrate deficiencies in previous attempts of detection of MPXV, and although multiple mismatches in the probe sequence were found in the majority of the other PXV species, there is only one mismatch present in Akhmeta virus, suggesting cross reaction with Akhmeta virus is highly likely. For the Akhmeta virus there is a single mismatch in the probe binding region. Given this G2R probe is a Taqman (linear) probe with 30 nt, 1/30 mismatch is insufficient to block probe binding, and there will be a cross reaction with Akhmeta virus using this assay. Such cross reactions are avoided by the present invention;

FIG. 2 shows a graph representing design of the MPX HA assay;

FIG. 3 shows a BLAST analysis of the amplification region (174 bp) showing highest homology with other non-MPX orthopoxviruses is from vaccinia virus (93.33% or lower identity), followed by horsepox virus (92.78% identity);

FIG. 4 illustrates the MPX-HA assay evaluated against a panel of genomic DNA from MPXV, vaccina virus, cowpox virus, camelpox virus and human;

FIGS. 5A-5B show analytical sensitivity of MPX-HA assay evaluated using both vector plasmid and monkeypox genomic DNA templates;

FIGS. 6A-6C illustrates design of the MPX-F3L assay;

FIG. 7 shows BLAST analysis of the F3L amplicon against non-MPX orthopoxviruses database. Top 20 sequences with highest identity are shown;

FIG. 8 shows specificity evaluation of MPX-F3L assay, using genomic DNAs from monkeypox, cowpox, camelpox, vaccinia and human. NTC is abbreviation of no template control. Human genomic DNA is purchased from Sigma (cat #11691112001), the rest DNAs are obtained from BEI resources; and

FIGS. 9A-9B shows analytical sensitivity evaluation of MPX-F3L qPCR assay using serial dilutions of (FIG. 9A) synthetic DNA oligos or (FIG. 9B) MPXV genomic DNA (BEI resources). FIG. 9A shows MPX-F3L assay detects as low as 25 copies of synthetic DNA oligos per reaction with a linear quantification efficiency of 93%. FIG. 9B shows consistent linear quantification features in detecting authentic MPXV genomic DNA, with a lower limit of detection at 50 genomes/reaction.

### DETAILED DESCRIPTION

The invention includes, according to certain embodiments, systems and processes relates to a specific assay, method, and kit used to detect MPXV without the risk of cross reaction and in real time, unlike current state of the art detection methods and antibody/antigen detection methods. There are numerous ways to detect MPXV at the molecular level using appropriate patient specimens. While any type of body fluid might be used, blood in particular, typically was not found to contain high level of the MPXV virus. Below are a few examples of previous attempts to detect MPXV and description of how the present invention overcomes the specificity and cross-reaction issues that plague these prior attempts to detect MPXV.

### Introduction and Prior Attempts

Sample sensitivity for detecting MPXV further presents challenges when using blood samples for real-time polymerase chain reaction (rt-PCR) testing. However, the present invention overcomes this challenge, and other obstacles as further described herein. Lesions exudate on a swab or crust are considered as the best and least invasive patient samples (McCollum and Damon, 2014). However, if testing for antigens, then these lesions have already formed, and an onset of the disease has begun. Earlier detection of MPXV using rt-PCR is preferred to possibly avoid lesion formation and other symptoms of the MPXV.

An example of prior antigen testing is called specific peptide-based rapid antigen test (RAT). While RAT might be easy and quick, it might not be specific to MPXV due to high molecular similarities among numerous orthopoxviruses (McCollum and Damon, 2014). Despite this, RAT is being utilized for the specific detection of MPXV (JOYSBIO, 2022). However, this type of antigen testing has specificity issues that the present invention avoids. In addition, while RAT might be useful for quick and largescale screening/diagnostics as in COVID-19, it is found to have considerable drawback due to its high false negative rate when the virus load is low (Peeling et al., 2021; Scohy et al., 2020).

Alternatively, the virus-specific antibodies can also be detected in patient samples. The presence of anti-Orthopoxvirus immunoglobulin M (IgM) antibodies indicate a recent exposure to Orthopoxvirus or it might also be due to a smallpox vaccination. This may cause confusion in the test results. It is also required to use this type of antibodies diagnostic test for Orthopoxvirus infection if prior smallpox vaccination was not so recent, perhaps up to six months (McCollum and Damon, 2014; Usme-Ciro et al., 2017). On the other hand, the presence of anti-Orthopoxvirus IgG antibodies may also indicate a previous (and not so recent) exposure to Orthopoxvirus or smallpox vaccination. This result further dissuades the use of antibodies and antigen diagnostic testing for MPXV.

There are numerous variants of PCR-based methods for the detection of Orthopoxvirus/MPXV genomic targets. Some examples are given in Table 3 further shown herein. Over 90 primer/probe sets were previously explored to target poxvirus genes. Ropp et al. (1995) used conventional PCR to amplify near-full-length hemagglutinin (HA, also known as B2R) gene and endonuclease digest electropherograms to distinguish 10 species of orthopoxviruses, including North American orthopoxviruses and MPXV. However, given the close similarities among sequences, primers were not exclusive to species and thus cross-hybridized to multiple members of the genus. Further, the use of multiple endonuclease cleavage profiles overly complicated the species identification. In addition, no attempts were made to distinguish different clades/strains of MPXV.

Table III illustrates various past used primer sets with the HA gene being the most popular target used for the detection of orthopox viruses (Aitichou et al., 2005; 2008; Damaso et al., 2000; Edghill-Smith et al., 2005; Espy et al., 2002; Ibrahim et al., 2003; Kulesh et al., 2004; Putkuri et al., 2009; Ropp et al., 1995). Yet, due to its high sequence similarity among different Orthopoxvirus members, the HA gene is commonly not used for the specific detection of MPXV or its clades/strains. For example, an rt-PCR primer and probe set used by Edghill-Smith et al. (2005) can detect over a dozen orthopoxviruses.

However, cross reaction to other pox virus such as cowpox is possible. The present invention overcomes this and other challenges by using a specific assay that was shown to give specific real-time detection of MPXV without cross reaction and better specificity than previously known assays. The rt-PCR is a standard method for the universal detection of poxviruses (Luciani et al., 2021). Numerous variants of the method—for example—multiplexed PCR, assay based on dried PCR reagent, etc. have been tried and tested by others. Combination of primers and/or probes were also used.

For example, only variola virus was specifically detected by a FAM (6-carboxyfluorescein)-labelled probe while camelpox, cowpox, monkeypox, and vaccinia viruses were detected by a TET (6-carboxytetramethylrhodamine)-labelled probe in a single PCR reaction (Aitichou et al., 2008). Davi et al. (2019) used recombinase polymerase amplification (RPA) assay targeting the G2R (=J2R) gene for the specific and rapid detection of MPXV within 3 to 10 minutes. The RPA-assay was claimed to be highly sensitive with a limit of detection of 16 DNA molecules/μl.

Prior art attempts to use TNFR (also known as J2R) gene in particular seems to be popular for the specific detection of MPXV and its two (Congo and west-African) clades. However, there are many draw backs including cross-reaction issues to detecting other virus other than the MPXV strain.

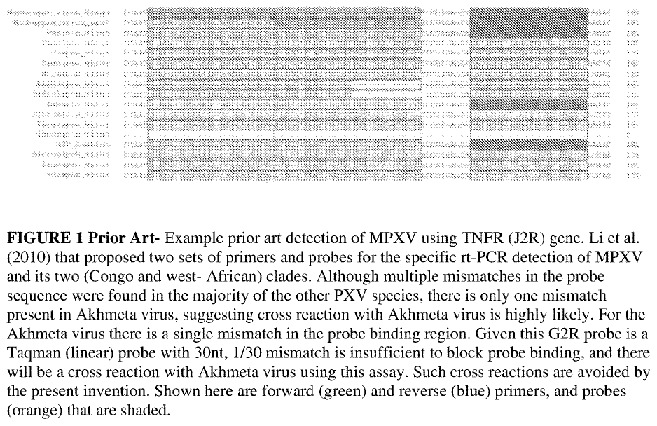

For example, Li et al. (2010) used two sets of rt-PCR primers and probes. While the first set of primers/probes would potentially detect MPXV over other PXVs (FIG. 1), a second set of primers/probes seemed specific for west-African MPXV due to a three-nucleotide insertion in the sequence. C3L/D14L gene was another target using either loop-mediated isothermal amplification (LAMP) (Iizuka et al., 2009) or rt-PCR method (Li et al., 2010). Although multiple mismatches in the probe sequence were found in the majority of the other PXV species, there is only one mismatch present in Akhmeta virus, suggesting cross reaction with Akhmeta virus is highly likely. For the Akhmeta virus there is a single mismatch in the probe binding region. Given this G2R probe is a Taqman (linear) probe with 30 nt, 1/30 mismatch is insufficient to block probe binding, and there will be a cross reaction with Akhmeta virus using this assay. Such cross reactions are avoided by the present invention. Shown here are forward (green) and reverse (blue) primers, and probes (orange) that are shaded. In addition, it seems that C3L/D14L is completely missing in the west-African clade of MPXV. However, as many OPV members such as vaccinia virus (VACV) and variola virus (VARV) have a highly similar C3L/D14L sequence, it would be very misleading to use C3L/D14L as a distinguishing target for the detection of the Congo clade of MPXV. The Pan American Health Organization (PAHO) interim guidance on laboratory testing for monkeypox virus mentions that while commercial PCR kits for detecting OPV and MPXV in particular are under development, no validated PCR kits are available in the market at present.

PAHO/WHO currently follow two protocols for OPV and MPXV detection (PAHO/WHO, 2022). The first one involves subjecting the samples (lesion swabs, vesicular fluids, and crusts) from suspected cases to OPV rt-PCR and the positive samples are subjected to MPXV-specific rt-PCR.

In the second one, the samples are directly subjected to MPXV-specific rt-PCR followed by differentiation of Congo and west-Africa clades. The PAHO/WHO uses primers/probes specific for G2R (=J2R) and C3L/D14L genes that have several issues of cross reaction and lack of specificity as previously discussed.

### Selection of Target Gene for MPXV

The MPXV genome has at least 190 open reading frames of ≥60 amino acid residues each (Shchelkunov et al., 2002). As shown in Table III, many genes have been used as likely targets. The use and selection of a target gene for the specific detection of MPXV is important, and complicated. The sequence in the central region of the MPXV genome, which encodes essential enzymes and structural proteins, is 96.3% identical to VARV. On the contrary, MPXV and VARV are said to have considerable differences in the regions encoding virulence and host-range factors near the ends of the genome (Shchelkunov et al., 2001).

While highly similar sequences lead to nonspecific primer/probe binding and lead to ambiguous detection of OPVs, highly divergent or variable sequence regions pose considerable challenge to detection. As in other viruses (Rao et al., 2021), comparison among OPVs such as VACV and VARV revealed that indels of 3-25 bp are common events in poxviruses (Coulson and Upton, 2011). The A27L gene is one such hypervariable target containing variations including truncations, deletions, insertions, and base changes in the two clades of MPXV (Meyer et al., 1997). The C3L/D14L gene is completely missing in west-African clade (Li et al., 2010). While these targets are often used to differentiate Congo and west-African MPXV clades (Saijo et al., 2008), given the inherent issues in the molecular detection, a negative result should not be taken as a confirmation.

In addition, poxviruses can undergo rapid changes as VACV, and was shown to acquire 7-10% increase in genome size via K3L gene amplification (Elde et al., 2012). There is considerable differences among MPXV clades (Kugelman et al., 2014). Further, like in all viruses, the evolution of the genome due to ongoing point-mutations is leading to additional challenges in detection. Compared to RNA viruses such as Poliovirus-1 that has a very high rate of 0.01 substitutions per site per year, the MPXV has a far lower rate of 7×10⁻⁷ (Stern and Andino, 2016).

Different MPXV/OPV sequences contain or can acquire a considerable number of substitutions, as shown in FIG. 1, which may result in undesirable outcomes. For example, primers with one or more mismatches can bind to templates and effect PCR amplification by <1.5 to >7 cycle threshold (Stadhouders et al., 2010). The substitutions are also problematic in RFLP based detection (Ropp et al., 1995). FIG. 1 shows an example prior art detection of MPXV using TNFR (J2R) gene. Li et al. (2010) that proposed two sets of primers and probes for the specific rt-PCR detection of MPXV and its two (Congo and west-African) clades. Although multiple mismatches in the probe sequence were found in the majority of the other PXV species, there is only one mismatch present in Akhmeta virus, suggesting cross reaction with Akhmeta virus is highly likely. For the Akhmeta virus there is a single mismatch in the probe binding region. Given this G2R probe is a Taqman (linear) probe with 30 nt, 1/30 mismatch is insufficient to block probe binding, and there will be a cross reaction with Akhmeta virus using this assay. Such cross reactions are avoided by the present invention. Shown here are forward (green) and reverse (blue) primers, and probes (orange) that are shaded.

### 1. MPX-HA Assay

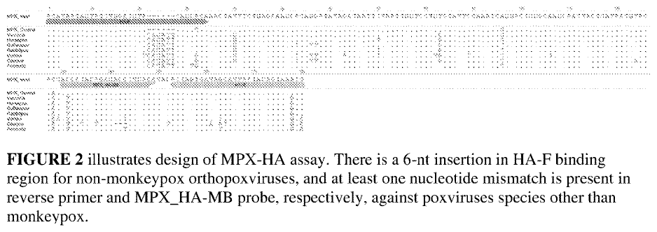

FIG. 2 illustrates an exemplary design of MPX-HA assay. There is a 6-nt insertion in HA-F binding region for non-monkeypox orthopoxviruses, and at least one nucleotide mismatch is present in reverse primer and MPX_HA-MB probe, respectively, against poxviruses species other than monkeypox.

The MPX-HA assay is developed using hemagglutinin (HA) gene as the detection target. The HA gene is highly conserved across the orthopoxvirus genus, with an overall ˜98% homology between different virus species. Therefore, HA has been used as the detection target for the pan-orthopoxvirus diagnostic real-time PCR assay developed earlier by USAMRIID (Kulesh et al. J Clin Microbiol. 2004 February; 42(2):601-9).

Unlike the pan-orthopox assay, described herein is a developed and novel MPX-HA real-time PCR assay with an intention to identify monkeypox virus highly selectively. With careful design, utilized herein is a pair of primers to amplify a 174-bp region of HA gene, and a MB probe to detect the amplicon. This HA MB probe has a perfect match (100%) with the target DNA sequence from monkeypox virus, and it has at least one nucleotide mismatch with other non-monkeypox orthopoxviruses.

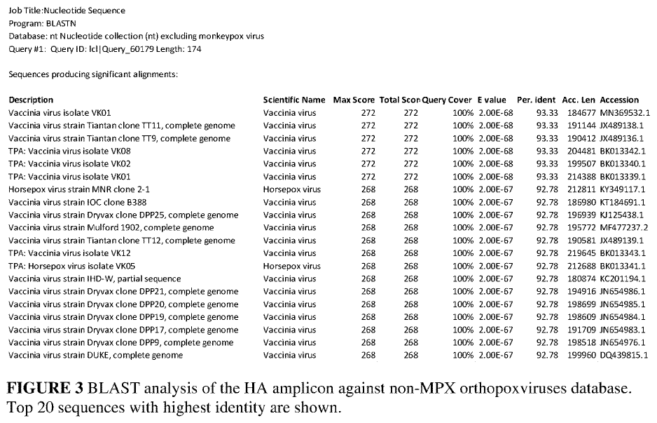

FIG. 3 illustrates a BLAST analysis of the HA amplicon against non-MPX orthopoxviruses database. Top 20 sequences with highest identity are shown in FIG. 3. As shown in FIG. 3, Blast analysis found that the highest sequence homology of the amplified region comes from vaccina virus (93.33% identity) followed by horsepox virus (92.78% identity), with only one mismatch in the reverse primer and one mismatch in the beginning of the loop sequence of the probe. A 6-nt insertion in forward primer (FIG. 3) is commonly seen with many non-monkeypox PXV, which seems to be sufficient to make PCR amplification highly selectively for MPXV. However, the conventional real-time MPX-HA PCR detects genomic DNA of both monkeypox virus and vaccinia virus, although cross-reaction with other orthopoxvirus species is not observed or expected based on in silico analysis. Based on the high sequence similarity of the amplified region among vaccinia, horsepox, buffalopox and rabbitpox viruses, similar cross reaction with these poxviruses species is highly likely.

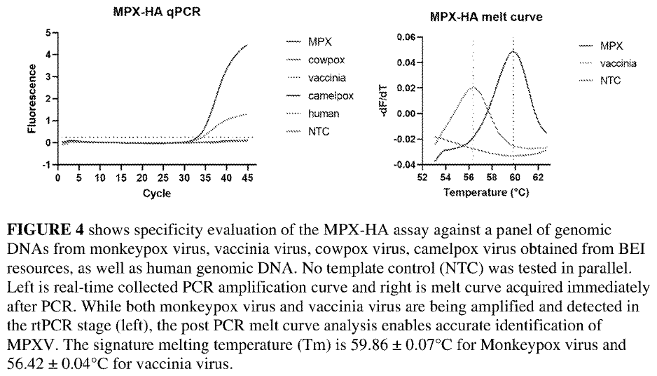

To overcome the specificity issue encountered in the regular real-time MPX-HA PCR, developed and described herein is a novel asymmetric real-time PCR and melt-curve analysis based MPX-HA assay. FIG. 4 illustrates specificity evaluation of the MPX-HA assay against a panel of genomic DNAs from monkeypox virus, vaccinia virus, cowpox virus, camelpox virus obtained from BEI resources, as well as human genomic DNA. No template control (NTC) was tested in parallel. Left is real-time collected PCR amplification curve and right is melt curve acquired immediately after PCR. While both monkeypox virus and vaccinia virus are being amplified and detected in the rtPCR stage (left), the post PCR melt curve analysis enables accurate identification of MPXV. The signature melting temperature (Tm) is 59.86±0.07° C. for Monkeypox virus and 56.42±0.04° C. for vaccinia virus.

As shown in FIG. 4, specificity of the MPX-HA assay was evaluated against a panel of genomic DNAs from monkeypox, vaccinia, cowpox, camelpox viruses. The asymmetric real-time PCR component of this new MPX-HA assay keeps the quantitative property of the detection, while the post-amplification melt-curve enables accurate differentiation of monkeypox virus from vaccinia virus. The assay is setup in a 20 μl reaction consisting of 10 μl of Premix Ex Taq (Probe qPCR) (TaKaRa Cat #RR390L), 0.8 μl of 1 μM forward primer, 2 μl of 10 μM reverse primer, 0.4 μl of 5 μM MPX-HA MB probe, and 5 μl of DNA template. Reactions are running on the Bio Molecular System mic qPCR cycler, with thermal profile of 95° C. for 2 min, 50 cycles of 95° C. for 5 sec and 56° C. for 20 sec, followed by 95° C. for 20 sec, then melted from 53° C. to 63° C. with a ramp rate of 0.1° C./s. This assay takes 1 hour and 7 min to complete. In the rtPCR phase, except vaccinia virus, no cross-reaction was observed for other non-monkeypox viruses tested or human DNA. However, MPXV is clearly differentiated from vaccinia virus by the post PCR melt curve analysis, with signature melting temperatures (Tm) of 59.86±0.07° C. for Monkeypox virus and 56.42±0.04° C. for vaccinia virus.

Specificity of the MPX-HA assay was evaluated against a panel of genomic DNAs from monkeypox virus, vaccinia virus, cowpox virus, camelpox virus obtained from BEI resources, as well as human genomic DNA purchased from Sigma.

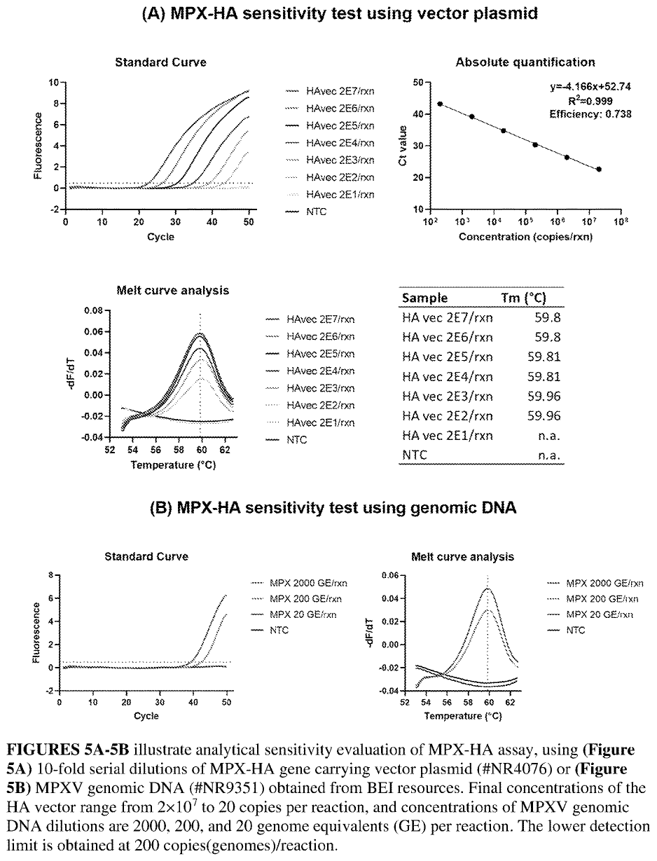

FIGS. 5A-B illustrate analytical sensitivity evaluation of MPX-HA assay, using (FIG. 5A) 10-fold serial dilutions of MPX-HA gene carrying vector plasmid (#NR4076) or (FIG. 5B) MPXV genomic DNA (#NR9351) obtained from BEI resources. Final concentrations of the HA vector range from 2×10⁷ to 20 copies per reaction, and concentrations of MPXV genomic DNA dilutions are 2000, 200, and 20 genome equivalents (GE) per reaction. The lower detection limit is obtained at 200 copies(genomes)/reaction.

As shown in FIGS. 5A-5B, the analytical sensitivity of the MPX-HA assay was evaluated using both vector plasmid carrying MPXV HA gene (BEI resources, NR4076) and monkeypox genomic DNA templates. The linear quantification range of the assay has at least 6-log magnitude with a limit of detection (LOD) at 200 copies of target DNA per reaction.

The following Table I further describes the MPX-HA assay. The following is one example given merely to demonstrate the inventive subject matter, and is not meant to limit the inventive subject matter to this one embodiment. Again, the MPX-HA assay may or may not be used in combination with the MPX-F3L assay, depending on the embodiment, to detect MPXV virus using the principles of the present invention.

TABLE I
MPX-HA assay

| Oligo | function | sequence (5′-3′) |
|---|---|---|
| HA-F | forward primer | ACATAATACTATCTGGATCTACACCRGA |
| | | [SEQ. 1] |
| HA-R | reverse primer | CATTTACTGTATTAATGCTATCACTAGT [SEQ. 2] |
| MPX_HA-MB | probe | FAM- |
| | | CGACGTACCATACAGACACCGTCACAACGTCG- |
| | | Dabcyl [SEQ. 3] |

MPX-HA assay setup

| | Vol (μl) |
|---|---|
| Premix Ex Taq (2X) | 10 |
| F Primer (1 μM) | 0.8 |
| R Primer (10 μM) | 2 |
| Probe (5 uM) | 0.4 |
| H2O | 1.8 |
| DNA template | 5 |
| Total | 20 |

Thermal Cycling

| | | |
|---|---|---|
| PCR | 95° C. 2 min | |
| | 95° C. 5 sec | × 50 |
| | 56° C. 20 sec | cycles |
| Melt | 95° C. 20 sec | |
| curve | 53º C. 20 sec | |
| | melt from 53° C. | |
| | 0.1º C./sec | |

2. Combination of MPX-HA Assay with MPX-F3L Assay

The MPX-F3L assay targets monkeypox virus F3L gene encoding the interferon (IFN) resistance factor, an important virulence factor of the virus. F3L was the target gene of one real-time PCR assay developed by the US Army Medical Research Institute of Infectious Diseases (USAMRIID) which was aimed to be MPX specific (Kulesh et al. Lab Invest. 2004 September; 84(9), 1200-8).

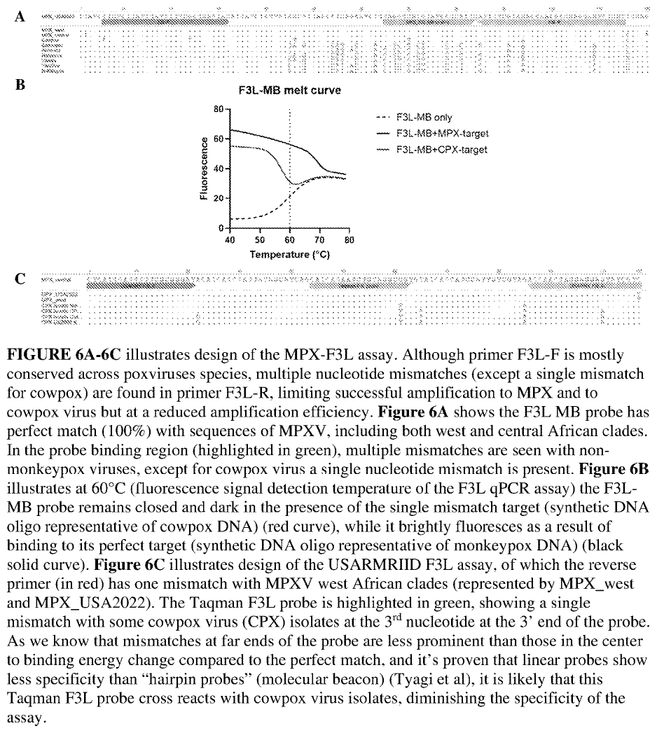

The 3′-minor groove binder (MGB) TaqMan probe was used in this USARMRIID assay for specific MPX detection. However, in silico analysis showed that a single mismatch at the far 3′end of the Tagman-MGB F3L probe with sequences from more recent cowpox virus isolates is likely to create cross-reaction with cowpox virus, diminishing the specificity of this earlier developed diagnostic assay as shown in FIG. 6C.

Adverting to FIGS. 6A-6C, illustrated are FIG. 6A that illustrates design of the MPX-F3L assay. Although primer F3L-F is mostly conserved across poxviruses species, multiple nucleotide mismatches (except a single mismatch for cowpox) are found in primer F3L-R, limiting successful amplification to MPX and to cowpox virus but at a reduced amplification efficiency. The F3L MB probe has perfect match (100%) with sequences of MPXV, including both west and central African clades. In the probe binding region (highlighted in green), multiple mismatches are seen with non-monkeypox viruses, except for cowpox virus a single nucleotide mismatch is present. FIG. 6B illustrates at 60° C. (fluorescence signal detection temperature of the F3L qPCR assay) the F3L-MB probe remains closed and dark in the presence of the single mismatch target (synthetic DNA oligo representative of cowpox DNA) (red curve), while it brightly fluoresces as a result of binding to its perfect target (synthetic DNA oligo representative of monkeypox DNA) (black solid curve). FIG. 6C illustrates design of the USARMRIID F3L assay, of which the reverse primer (in red) has one mismatch with MPXV west African clades (represented by MPX_west and MPX_USA2022). The Tagman F3L probe is highlighted in green, showing a single mismatch with some cowpox virus (CPX) isolates at the 3^{rd} nucleotide at the 3′ end of the probe. As we know that mismatches at far ends of the probe are less prominent than those in the center to binding energy change compared to the perfect match, and it's proven that linear probes show less specificity than “hairpin probes” (molecular beacon) (Tyagi et al), it is likely that this Taqman F3L probe cross reacts with cowpox virus isolates, diminishing the specificity of the assay.

Given that F3L homology between monkeypox and cowpox viruses is as high as 97%, the present invention includes a newly created, molecular beacon (MB) based F3L real-time PCR assay, taking advantage of the single nucleotide mismatch (SNP) recognition feature of MBs. This assay is composed of a forward primer, a reverse primer generating a 110-bp amplicon, and a F3L MB probe for MPX-specific detection.

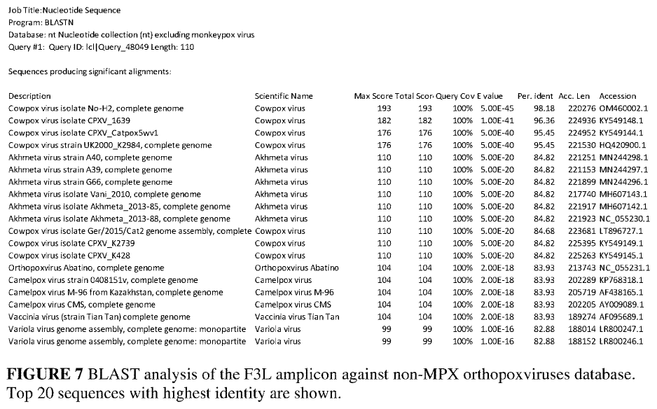

FIG. 7 shows the BLAST analysis of the F3L amplicon against non-MPX orthopoxviruses database. Top 20 sequences with highest identity are shown. Illustrated by FIG. 7, BLAST analysis of the amplification region (110 bp) shows that the highest homology with other non-MPX orthopoxviruses is from the cowpox virus (95.45˜98.18% identity), followed by Akhmeta virus (84.82% identity) and camelpox virus (83.93% identity).

However, design of the MPX-specific F3L MB probe ensures that at the selected annealing temperature, the probe remains closed and dark in the presence of the single mismatch target, while it tightly binds to its perfect target resulting in bright fluorescence as a detection signal (FIG. 6B).

The MPX-F3L assay is setup in a 20 μl reaction consisting of 10 μl of Premix Ex Taq (Probe qPCR) (TaKaRa Cat #RR390L), 0.4 μl of 10 μM forward primer, 0.4 μl of 10 μM reverse primer, 0.4 μl of 5 μM MPX-F3L MB probe, and 5 μl of DNA template. Reactions are running on the Bio Molecular System mic qPCR cycler, with thermal profile of 95° C. for 2 min followed by 40 cycles of 95° C. for 5 sec and 60° C. for 20 sec.

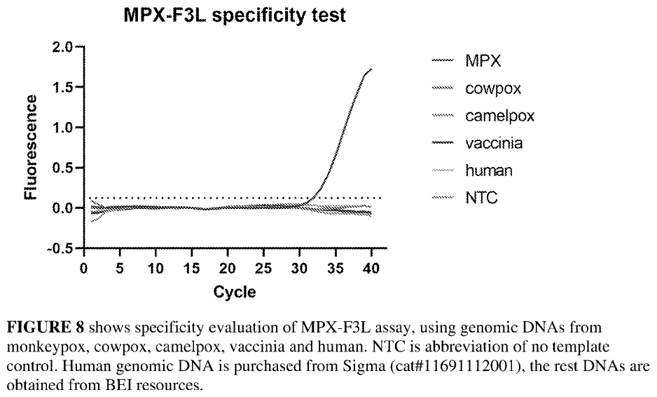

FIG. 8 shows specificity evaluation of MPX-F3L assay, using genomic DNAs from monkeypox, cowpox, camelpox, vaccinia and human. NTC is abbreviation of no template control. Human genomic DNA is purchased from Sigma (cat #11691112001), the rest DNAs are obtained from BEI resources.

The real-time test takes 48 min to complete. As shown in FIG. 8, specificity of the MPX-F3L assay was evaluated against a panel of genomic DNAs from monkeypox virus, vaccinia virus, cowpox virus, camelpox virus obtained from BEI resources, as well as human genomic DNA purchased from Sigma (cat #11691112001). No cross-reaction was observed for any non-monkeypox viruses tested or human DNA.

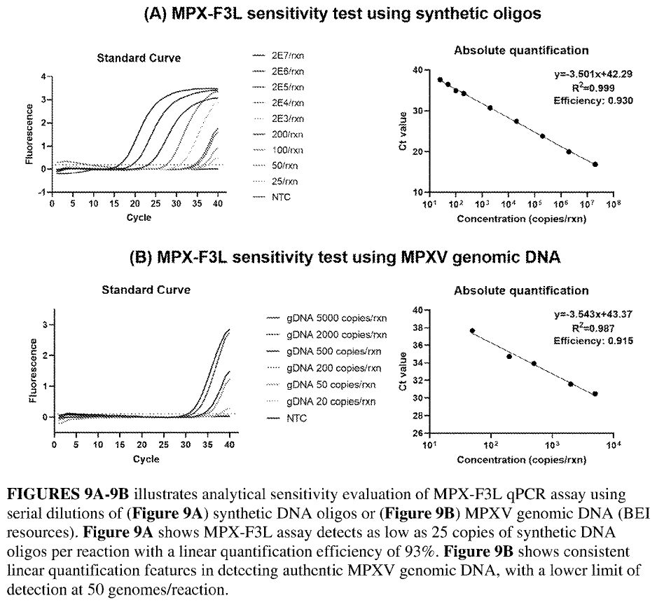

FIGS. 9A-9B illustrates analytical sensitivity evaluation of MPX-F3L qPCR assay using serial dilutions of (FIG. 9A) synthetic DNA oligos or (FIG. 9B) MPXV genomic DNA (BEI resources). FIG. 9A shows MPX-F3L assay detects as low as 25 copies of synthetic DNA oligos per reaction with a linear quantification efficiency of 93%. FIG. 9B shows consistent linear quantification features in detecting authentic MPXV genomic DNA, with a lower limit of detection at 50 genomes/reaction.

As shown in FIGS. 9A-9B, the analytical sensitivity of the MPX-F3L assay was evaluated using both synthetic oligo targets and monkeypox genomic DNA templates. The linear quantification range of the assay has 7-log magnitude with a limit of detection (LOD) at 25 copies of target DNA per reaction.

The following Table II further describes the MPX-F3L assay. The following is one example given merely to demonstrate the inventive subject matter, and is not meant to limit the inventive subject matter to this one embodiment.

TABLE II
MPX-F3L assay

| Oligo | function | sequence (5′-3′) |
|---|---|---|
| F3L-F | forward primer | CTCTCTCATTGATTTTTCGCGGGATAC [SEQ. 4] |
| F3L-R | reverse primer | ACGATACTCCTCCTCGTTGGTCTAC [SEQ. 5] |
| MPX_F3L-MB | probe | FAM- |
| | | CGCGATTAGGCCGTGTATCAGCATCCATCGCG- |
| | | Dabcyl [SEQ. 6] |

MPX-F3L assay setup

| | Vol (μl) |
|---|---|
| Premix Ex Taq (2X) | 10 |
| F Primer (10 μM) | 0.4 |
| R Primer (10 μM) | 0.4 |
| Probe (5 uM) | 0.4 |
| H2O | 3.8 |
| DNA template | 5 |
| Total | 20 |

Thermal Cycling

| | |
|---|---|
| 95° C. 2 min | |
| 95° C. 5 sec | × 40 |
| 60° C. 20 sec | cycles |

Given the limited access to both genomic DNA and culturable stock of different orthopoxvirus species, the present investigators only assessed the inventive assays using the currently available material. Expansion of the specificity testing panel may be possible for both assays. The low concentration of the genomic DNA of monkeypox obtained from BEI resources largely limited the sensitivity evaluation magnitude. The present investigators used either synthetic oligo targets or vector DNA as an alternative. In addition, it is recognized that the currently disclosed novel MPX-HA assay has limited sensitivity as compared to the disclosed novel MPX-F3L assay. The present investigators hypothesize that optimization of the presently disclosed novel MPX-HA assay to improve the sensitivity.

Any headings and sub-headings utilized in this description are not meant to limit the embodiments described thereunder. Features of various embodiments described herein may be utilized with other embodiments even if not described under a specific heading for that embodiment.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

TABLE III
List of Commonly Used Genes, Primers, and Probes in the Detection of
MPXV Having Limitations in Specificity and Cross-Reaction¹

| Gene | Forward and reverse primers (and probes) | Len | Assay | Reference |
|---|---|---|---|---|
| A4L | GTCAACGCTGGAAGGAGTG | 217 | rt-PCR | Kuo and |
| (CP) | CCAGCAGACAGCCTATCC | | | Wang, 2013 |
| | [CTCCTGTACTAAAACCACGWCAACAAACT] | | | |
| A27L | AATACAAGGAGGATCT | 1549 | PCR | Meyer |
| (ATI) | CTTAACTTTTTCTTTCTC | | | et al., 1997 |
| | GAGAGAATCTCTTGATAT | 601 | PCR | Neubauer, |
| | ATTCTAGATTGTAATC | | | et al., 1997 |
| | GAGATTAGCAGACTCCAA | 163 | rt-PCR | Saijo et |
| | GATTCAATTTCCAGTTTGTAC | | | al., 2008 |
| | [GCAGTCGTTCAACTGTATTTCAAGATCTGAGA | | | |
| | T/ | | | |
| | CTAGATTGTAATCTCTGTAGCATTTCCACGGC] | | | |
| | CCGTTACCGTTTTTACAATCGTTAATCAATGCT | — | LAMP | Iizuka |
| | GATATGGAAAAGAGA | | | et al., 2009 |
| | ATAGGCTAAAGACTAGAATCAGGGATTCTGAT | | | |
| | TCATCCTTTGAGAAG | | | |
| | TACAGTTGAACGACTGCG | 221 | | |
| | AGTTCAGTTTTATATGCCGAAT | | | |
| | GATGTCTATCAAGATCCATGATTCT | 115 | | |
| | TCTTGAACGATCGCTAGAGA | | | |
| | TGGAGTCTGCTAATCTCTGTAAGATTAGAGAA | — | | |
| | CTAGAGAATAAGTTGACC | | | |
| | TGAGTGAATGCCGTGGAAATGCGCAGTCGTTC | | | |
| | AACTGTA | | | |
| | CACAAGAAGTTGATGCACTG | 253 | | |
| | CAGCATTGATTTCATTATTACGT | | | |
| | CGCTCTCGATGCAGTC | 128 | | |
| | CAGAGATTACAATCTAGAATCTCAG | | | |
| A29L | CCAGAGATATCATAGCCGCTCTT | 156/157 | rt-PCR | Dumont |
| (14kd | GAAACTCTCAAACAACGRCTAACT | | | et al., 2014 |
| protein) | [TAAATAGAACGTCATCATT] | | | |
| B2R | ATGACACAATTACCAATAC | 942 | ORf | Ropp et |
| (HA) | CTAGACTTTGTTCTCTG | | | al., 1995 |
| | CTGATAATGTAGAAGAC | 406 | PCR | |
| | TTGTATTTACGTGGGTG | | | |
| | GATGATGCAACTCTATCATGTA | 131 | rt-PCR | Edghill- |
| | GTATAATTATCAAAATACAAGACGTC | | | Smith et |
| | [AGTGCTTGGTATAAGGAG] | | | al., 2005 |
| B6R | ATTGGTCATTATTTTTGTCACAGGAACA | 83 | rt-PCR | Li et al., |
| (EEV) | AATGGCGTTGACAATTATGGGTG | | | 2006 |
| | [AGAGATTAGAAATA] | | | |
| B7R | ACGTGTTAAACAATGGGTGATG | 99 | rt-PCR | Shchelkunov |
| | AACATTTCCATGAATCGTAGTCC | | | et al., 2011 |
| | [TGAATGAATGCGATACTGTATGTGTGGG] | | | |
| C3L/D14L | TGGGAGCATTGTAACTTATAGTTGCCCTCCTGA | — | LAMP | Iizuka |
| | ACACATGACA | | | et al., 2009 |
| | ATCCTCGTATCCGTTATGTCTTCCCACCTATTTG | | | |
| | CGAATCTGTT | | | |
| | TGGGTGGATTGGACCATT | 199 | | |
| | ATGGTATGGAATCCTGAGG | | | |
| | GATATTCGTTGATTGGTAACTCTGG | 117 | | |
| | GTTGGATATAGATGGAGGTGATTGG | | | |
| | TGTCTACCTGGATACAGAAAGCAA | 100 | rt-PCR | Li et al., |
| | GGCATCTCCGTTTAATACATTGAT | | | 2010 |
| | [CCCATATATGCTAAATGTACCGGTACCGGA] | | | |
| E9L | TCAACTGAAAAGGCCATCTATGA | 101 | rt-PCR | Li et al., |
| (DNA | GAGTATAGAGCACTATTTCTAAATCCCA | | | 2006 |
| Polymerase) | [CCATGCAATATACGTACAAGATAGTAGCCAAC] | | | |
| F3L | CTCATTGATTTTTCGCGGGATA | 107 | rt- | Kulesh |
| | GACGATACTCCTCCTCGTTGGT | | PC | et al., |
| | [CATCAGAATCTGTAGGCCGT] | | R | 2004 |
| | CATCTATTATAGCATCAGCATCAGA | 79 | rt- | Maksyu |
| | GATACTCCTCCTCGTTGGTCTAC | | PC | tov et |
| | [TGTAGGCCGTGTATCAGCATCCATT] | | R | al., 2015 |
| J2R | CACACCGTCTCTTCCACAGA | 82/85 | rt-PCR | Li et al., |
| (TNFR) | GATACAGGTTAATTTCCACATCG | | | 2010 |
| | [AACCCGTCGTAACCAGCAATACATTT] | | | |
| | GGAAAATGTAAAGACAACGAATACAG | 90 | rt-PCR | |
| | GCTATCACATAATCTGGAAGCGTA | | | |
| | [AAGCCGTAATCTATGTTGTCTATCGTGTCC] | | | |
| | AATAAACGGAAGAGATATAGCACCACATGCAC | 181 | RPA | Davi et |
| | GTGAGATGTAAAGGTATCCGAACCACACG | | | al., 2019 |
| | [ACAGAAGCCGTAATCTATGTTGTCTATCGQTF | | | |
| | CCTCCGGGAACTTA] | | | |
| N3R | AACAACCGTCCTACAATTAAACAACA | 139 | rt-PCR | Kulesh |
| | CGCTATCGAACCATTTTTGTAGTCT | | | et al., 2004 |
| | TATAACGGCGAAGAATATACT] | | | |

LAMP loop-mediated isothermal amplification, ORF open reading frame, RPA recombinase
polymerase amplification, rt-PCR real-time polymerase chain reaction.
¹Source: Ghate et al., 2022 Jul.

### REFERENCES

Kulesh et al. Lab Invest. 2004 September; 84(9), (1200-8)
Kulesh et al. J Clin Microbiol. 2004 February; 42(2):601-9
Ghate et al., Molecular detection of monkeypox and related viruses—Challenges and opportunities, OSF Preprints 2022 July; DOI 10.31219/osfio/jtdnz
Tyagi S, Bratu D P, Kramer F R. 1998. Multicolor molecular beacons for allele discrimination. Nat Biotechnol 16:49-53.
Aitichou M, Javorschi S, Ibrahim M S (2005). Two-color multiplex assay for the identification of orthopox viruses with real-time LUX-PCR. Molecular and Cellular Probes 19:323-328.
Aitichou M, Saleh S, Kyusungb P, et al. (2008). Dual-probe real-time PCR assay for detection of variola or other orthopoxviruses with dried reagents. Journal of Virological Methods 153:190-195.
Baker R E, Mahmud A S, Miller I F, et al. (2022). Infectious disease in an era of global change. Nature Reviews Microbiology 20:193-205.
Chen N, Li G, Liszewski M K, et al. (2005). Virulence differences between monkeypox virus isolates from west Africa and the Congo basin. Virology 340:46-63.
Coulson D, Upton C (2011). Characterization of indels in poxvirus genomes. Virus Genes 42:171-177.
Damaso C R, Esposito J J, Condit R C, et al. (2000). An emergent poxvirus from humans and cattle in Rio de Janeiro State: Cantagalo virus may derive from Brazilian smallpox vaccine. Virology 277:439-449.
Davi S D, Kissenkötter J, Faye M, et al. (2019). Recombinase polymerase amplification assay for rapid detection of monkeypox virus. Diagnostic Microbiology and Infectious Disease 95:41-45.
Doellinger J, Schaade L, Nitsche A (2015). Comparison of the cowpox virus and vaccinia virus mature virion proteome: Analysis of the species-and strain-specific proteome. PLoS ONE 10:e0141527.
Durski K N, McCollum A M, Nakazawa Y, et al. (2018). Emergence of monkeypox—west and central Africa, 1970-2017. MMWR Morbidity and Mortality Weekly Report 67:306-310.
Edghill-Smith Y, Golding H, Manischewitz J, et al. (2005). Smallpox vaccine-induced antibodies are necessary and sufficient for protection against monkeypox virus. Nature Medicine 11:740-747.
Elde N C, Child S J, Eickbush M T, et al. (2012). Poxviruses deploy genomic accordions to adapt rapidly against host antiviral defenses. Cell 150:831-841.
Eshoo M W, Whitehouse C A, Nalca A, et al. (2009). Rapid and high-throughput pan-Orthopoxvirus detection and identification using PCR and mass spectrometry. PLoS ONE 4:e6342.
Espy M J, Cockerill I I I FR, Meyer R F, et al. (2002). Detection of smallpox virus DNA by LightCycler PCR. Journal of Clinical Microbiology 40:1985-1988.
Grant R J, Baldwin C D, Nalca A, et al. (2010). Application of the Ibis-T5000 pan-orthopoxvirus assay to quantitatively detect monkeypox viral loads in clinical specimens from macaques experimentally infected with aerosolized monkeypox virus. American Journal of Tropical Medicine and Hygiene 82:318-323.
Huggins J, Goff A, Hensley L, et al. (2009). Nonhuman primates are protected 469 from smallpox virus or monkeypox virus challenges by the antiviral drug ST-246. Antimicrobial Agents and Chemotherapy. 53:2620-2625.
Ibrahim S M, Kulesh D A, Saleh S S, et al. (2003). Real-time PCR assay to detect smallpox virus. Journal of Clinical Microbiology 41:3835-3839.
Iizuka I, Saijo M, Shiota T, et al. (2009). Loop-mediated isothermal amplification-based diagnostic assay for monkeypox virus infections. Journal of Medical Virology 81:1102-1108.
JOYSBIO (2022). Monkeypox rapid test kit (https://en.joysbio.com/monkeypox-rapid-test-kit/, last accessed on 20-06-2022).
Karem K L, Reynolds M, Braden Z et al. (2005). Characterization of acute-phase humoral immunity to monkeypox: Use of immunoglobulin M enzyme-linked immunosorbent assay for detection of monkeypox infection during the 2003 north American outbreak. Clinical and Diagnostic Laboratory Immunology 12:867-872.
Kugelman J R, Johnston S C, Mulembakani P M, et al. (2014). Genomic variability of monkeypox virus among humans, Democratic Republic of the Congo. Emerging Infectious Diseases 20:232-239.
Kulesh D A, Loveless B M, Norwood D, et al. (2004). Monkeypox virus detection in rodents using real-time 3′-minor groove binder TaqMan® assays on the Roche LightCycler. Laboratory Investigation 84:1200-1208.
Learned L A, Reynolds M G, Wassa D W, et al. (2005). Extended interhuman transmission of monkeypox in a hospital community in the Republic of the Congo, 2003. American Journal of Tropical Medicine and Hygiene 73:428-434.
Li Y, Olson V A, Laue T, et al. (2006). Detection of monkeypox virus with real-time PCR assays. Journal of Clinical Virology 36:194-203.
Li Y, Zhao H, Wilkins K, et al. (2010). Real-time PCR assays for the specific detection of monkeypox virus West African and Congo Basin strain DNA. Journal of Virological Methods 169:223-227.
Likos A M, Sammons S A, Olson V A, et al. (2005). A tale of two clades: Monkeypox viruses. Journal of General Virology 86:2661-2672.
Luciani L, Inchauste L, Ferraris O, et al. (2021). A novel and sensitive real-time PCR system for universal detection of poxviruses. Scientific Reports 11:1798.
Manes N P, Estep R D, Mottaz H M, et al. (2008). Comparative proteomics of human monkeypox and vaccinia intracellular mature and extracellular enveloped virions. Journal of Proteome Research 7:960-968.
McCollum A M, Damon I K (2014). Human monkeypox. Clinical Infectious Diseases 58:260-267.
McMullen C L (2015). A one health perspective on disease dynamics: 519 Human monkeypox transmission in Sankuru district, Democratic Republic of Congo. Duke University ProQuest Dissertations Publishing 1587691.
Meyer H, Ropp S L, Esposito J J (1997). Gene for A-type inclusion body protein is useful for a polymerase chain reaction assay to differentiate orthopoxviruses. Journal of Virological Methods 64:217-221.
Neubauer H, Reischl U, Ropp S, et al. (1998). Specific detection of monkeypox virus by polymerase chain reaction. Journal of Virological Methods 74:201-207. OWID (2022). Monkeypox data explorer (https://ourworldindata.org/explorers/monkeypox?tab=table&time=2022-05-24..latest&facet=none&Metric=Confirmed+cases&Frequency=Cumulative&Shown+by=Dat e+o f+confirmation&country=˜OWID_WRL, last accessed on 24-06-2022).
PAHO/WHO (2022). Laboratory guidelines for the detection and diagnosis of monkeypox virus infection (https://www.paho.org/en/documents/laboratory-guidelines-detection-and-diagnosis monkeypox-virus-infection, last accessed on 24-06-2022).
Parker S, Nuara A, Buller R M L, Schultz D A (2007). Human monkeypox: An emerging zoonotic disease. Future Microbiology 2:17-34.
Peeling R W, Olliaro P L, Boeras D I, Fongwen N (2021). Scaling up COVID-19 rapid antigen tests: promises and challenges. Lancet Infectious Diseases 21:e290-e295.
Putkuri N, Piiparinen H, Vaheri A, et al. (2009). Detection of human orthopoxvirus infections and differentiation of smallpox virus with real-time PCR. Journal of Medical Virology 81:146-152.
Rao R S P, Ahsan N, Xu C, et al. (2021). Evolutionary dynamics of indels in SARS-CoV-2 spike glycoprotein. Evolutionary Bioinformatics 17:1064616.
Reed K D, Melski J W, Graham M B, et al. (2004). The detection of monkeypox in humans in the western hemisphere. The New England Journal of Medicine 350:342-350.
Ropp S L, Jin Q, Knight J C, et al. (1995). PCR strategy for identification and differentiation of small pox and other orthopoxviruses. Journal of Clinical Microbiology 33:2069-2076.
Saijo M, Ami Y, Suzaki Y, et al. (2008). Diagnosis and assessment of monkeypox virus (MPXV) infection by quantitative PCR assay: Differentiation of Congo Basin and West African MPXV strains. Japanese Journal of Infectious Diseases 61:140-142.
Scohy A, Anantharajah A, Bodéus M, et al. (2020). Low performance of rapid antigen detection test as frontline testing for COVID-19 diagnosis. Journal of Clinical Virology 129:104455.
Shchelkunov S N, Totmenin A V, Babkin I V, et al. (2001). Human monkeypox and smallpox viruses: Genomic comparison. FEBS Letters 509:66-70.
Shchelkunov S N, Totmenin A V, Safronov P F, et al. (2002). Analysis of the monkeypox virus genome. Virology 297:172-194.
Sklenovská N, Van Ranst M (2018). Emergence of monkeypox as 571 the most important orthopoxvirus infection in humans. Frontiers in Public Health 6:241.
Stadhouders R, Pas S D, Anber J, et al. (2010). The effect of primer-template mismatches on the detection and quantification of nucleic acids using the 5′ nuclease assay. Journal of Molecular Diagnostics 12:109-117.
Stern A, Andino R (2016). Viral evolution—It is all about mutations. In Katze M G, et al. (Eds.) Viral pathogenesis—From basics to systems biology. PP 233-240 (doi:10.1016/B978-0-12-800964-2.00017-3).
Usme-Ciro J A, Paredes A, Walteros D M, et al. (2017). Detection and molecular characterization of zoonotic poxviruses circulating in the Amazon region of Colombia, 2014. Emerging Infectious Diseases 23:649-653.
WHO (2022a). Multi-country monkeypox outbreak in non-endemic countries (https://www.who.int/emergencies/disease-outbreak-news/item/2022-DON385, last accessed on May 30, 2022).
WHO (2022b). Multi-country monkeypox outbreak: situation update (https://www.who.int/emergencies/disease-outbreak-news/item/2022-DON393 #:˜:text=Description%20of%20the%20outbreak,May%202022%20(Figure%201), last accessed on 23-06-2022).
Woolhouse M, Scott F, Hudson Z, et al. (2012). Human viruses: Discovery and emergence. Philosophical Transactions of the Royal Society B: Biological Sciences 367:2864-2871.

What is claimed is:

1. An assay composition for detecting a monkeypox virus (MPXV), comprising:
a primer pair and a probe;
wherein the primer pair includes a forward primer and a reverse primer having the sequences in [SEQ.1] and [SEQ.2], respectively; and
the probe includes the sequence in [SEQ.3] for increasing specificity and reducing cross-reaction as compared to conventional rt-PCR assays.

2. The composition according to claim 1, wherein the probe is characterized in that: 5′ end of the probe is marked with a fluorescent reporter group FAM, and 3′ end is marked with a fluorescent quenching group Dabcyl.

3. The composition according to claim 1, wherein the primer pair has a concentration in 0.8 μl of 1 μM for the forward primer, and 2 μl of 10 μM for the reverse primer, and the probe has a concentration in 0.4 μl of 5 μM.

4. The composition according to claim 1, wherein the probe is a MPX-specific HA MB probe that ensures during the post PCR melt curve analysis it melts from the HA target amplified from MPXV at a temperature significantly higher than that from other orthopoxvirus species which may be likely amplified by the same PCR.

5. The composition according to claim 4, further including a second assay MPX-F3L having a second primer pair and a second probe; wherein the second primer pair includes a second forward primer and a second reverse primer having the sequences in [SEQ.4] and [SEQ.5], respectively; and the second probe includes the sequence in [SEQ.6].

6. A test kit for detecting a monkeypox virus (MPXV), comprising the composition described in claim 1 or the compositions in claim 5, and following material: a 2× master mix for real-time PCR (qPCR); a PCR enzyme, anti-Taq antibody, a qPCR-optimized buffer; and a heat-resistant RNase enzyme.

7. The test kit according to claim 6, further includes a DNA template; wherein the DNA template is a 5 μl DNA template having a synthetic oligo target or a monkeypox genomic DNA template.

8. The test kit according to claim 7, wherein the test kit utilizes a DNA sample from a patient for the detection of MPXV prior to appearance of lesions or symptomatology of MPXV.

9. A method of using an assay in the detection of monkeypox virus (MPXV), comprising the steps of:
providing a MPX-HA assay includes setting up in the 20 μl reaction material comprising of 10 μl of Premix Ex Taq (Probe qPCR), 0.8 μl of 1 μM a forward primer [SEQ. 1], 2 μl of 10 μM a reverse primer [SEQ.2], 0.4 μl of 5 μM MPX-HA MB probe [SEQ. 3], and 5 μl of DNA template;
running a reaction on the qPCR cycler with thermal profile of 95° C. for 2 min, 50 cycles of 95° C. for 5 sec and 56° C. for 20 sec, followed by 95° C. for 20 sec, then melted from 53° C. to 63° C. with a ramp rate of 0.1° C./s; and
obtaining rt-PCR test results at least after 1 hour and 7 min to complete the reaction.

10. The method according to claim 9, further including the steps of:
preparing a MPX-F3L second assay that includes setting up in a 20 μl reaction material comprising of 10 μl of Premix Ex Taq (Probe qPCR), 0.4 μl of 10 μM second forward primer [SEQ.4], 0.4 μl of 10 μM second reverse primer [SEQ.5], 0.4 μl of 5 μM MPX-F3L MB second probe [SEQ.6], and 5 μl of DNA template;
running a second reaction on a qPCR cycler with a thermal profile of 95° C. for 2 min followed by 40 cycles of 95° C. for 5 sec and 60° C. for 20 sec; and
obtaining rt-PCR test results at least after 48 minutes to complete the second reaction.
